(19) United States
(12) Patent Application Publication
(10) Pub. No.: US 2009/0093865 A1
(43) Pub. Date: Apr. 9, 2009

(54) METHOD AND SYSTEM FOR CONTROLLING THE SPREAD OF MICROORGANISMS AMONG SUBJECTS IN A GROUP

(75) Inventors: Yosef Krespi, New York, NY (US); Abraham Kometz, New York, NY (US)

Correspondence Address: IP Patent Docketing;K&L GATES LLP, 599 Lexington Avenue, 33rd Floor, New York, NY, 10022-6030, US

(73) Assignee: VALAM INC., New York, NY (US)

(21) Appl. No.: 11/946,287

(22) Filed: Nov. 28, 2007

Related U.S. Application Data
(60) Provisional application No. 60/867,528, filed on Nov. 28, 2006.

(51) Int. Cl. A61N 5/06

(52) U.S. Cl. 607/88

(57) ABSTRACT

A method of controlling the spread of an infectious microorganism, for example methicillin-resistant Staphylococcus aureus (“MRSA”) in a group of subjects including people asymptomatic for infection with the microorganism. A dose of microorganism-reducing light can be applied to each anterior nasal cavity of each subject in the group. Optionally a colorant can be applied to the anterior nasal cavity to sensitize any infectious microorganisms present to the microorganism-reducing light. A light applicator system having a hollow light-transmissive nasal dilator insertable into a nostril and a light output member accommodatable in the hollow interior of the nasal dilator can be employed, optionally with a light diffuser around the light output member. One example of light applicator system comprises a hand piece removably attachable to an optical fiber coupled to a light source, the optical fiber constituting the light member.

### CROSS-REFERENCE TO A RELATED APPLICATION

This application claims the benefit of provisional patent application No. 60/867,528 filed Nov. 28, 2006.

### STATEMENT REGARDING FEDERALLY SPONSORED RESEARCH OR DEVELOPMENT

(Not applicable.)

### BACKGROUND OF THE INVENTION

The present invention relates to a method and system for controlling the spread of microorganisms between subjects in a group. More particularly, but not exclusively the invention relates to a method and system for controlling the spread of antibiotic-resistant microorganisms, for example methicillin-resistant Staphylococcus aureus.

Staphylococcus aureus (sometimes “S. aureus” hereinafter) is a virulent bacterium that colonizes the nasal vestibule adjacent the nares, or nasal openings, of significant numbers of adult humans, possibly as many as 25 percent of some populations. Nasal colonization with S. aureus is recognized as a risk factor for subsequent infection with S. aureus. The bacterium can cause illnesses ranging from minor skin infections and abscesses, to life-threatening diseases such as pneumonia, meningitis and endocarditis. Because of its life-threatening potential, cases of identified infection with S. aureus must usually receive aggressive treatment including rigorous antibiotic therapies and possibly including a stay in an intensive care unit. Furthermore, nasal carriage of S. aureus can pose a significant threat to surgical practice, as it may induce serious postoperative infections.

A still further problem presented by S. aureus is that the bacterium, like many others, has developed resistance to treatment with commonly-used antimicrobial agents such as methicillin. Because of the resultant difficulties in treating it, methicillin-resistant S. aureus(known as “MRSA”), can cause dangerous and costly infections. Furthermore, some people may be carriers for resistant strains of S. aureus, harboring populations of the bacterium yet exhibiting no significant symptoms of infection. Consequently, methicillin-resistant S. aureus may readily spread within a group of subjects and can be difficult to control.

Certain groups are especially at risk of developing a S. aureus-related disease. These include patients undergoing surgical procedures and patients with frequent breaks in the skin, such as those undergoing hemodialysis and/or continuous ambulatory peritoneal dialysis. It is known that nasal decolonization with mupirocin ointment can reduce the risk of infection in some high-risk groups. Also, it is known to use mupirocin with the intent of decolonizing carriers of methicillin-resistant S. aureus.

Mupirocin, however, may be ineffective against methicillin-resistant S. aureus. According to NDA 50-788, pp 3-7, updated by the FDA (United States Food and Drug Administration) Dec. 4, 2002 mupirocin, which may be contained in mupirocin ointment, is a naturally occurring antibiotic. Mupirocin is described in NDA 50-788 as an antibacterial agent produced by fermentation using the organism Pseudomonas florescens and its spectrum of activity includes gram-positive bacteria. NDA 50-788 furthermore states that methicillin resistance and mupirocin resistance commonly occur together in Staphylococcus areeus.

S. Harbarth et al. in Antimicrob Agents Chemother 1999 June; 43(6):1412-6, “Randomized, placebo-controlled, double-blind trial to evaluate the efficacy of mupirocin for eradicating carriage of methicillin-resistant Staphylococcus aureus.”, describe a randomized controlled trial of mupirocin versus a placebo wherein methicillin-resistant S. aureus was found to be successfully eradicated in only 44% of carriers.

A recent review of known decolonization methods for MRSA by M. Loeb et al. as described in “Antimicrobial drugs for treating methicillin-resistant Staphylococcus aureus colonization.” Cochrane Database Syst Rev 2003; (4):CD003340 concluded that there is insufficient evidence to support the efficacy of mupirocin for this purpose.

One skilled in the art will understand that, as is the case with other antimicrobial agents, resistance to mupirocin can develop in environments where it is extensively used. Resistance rates to mupirocin of greater than fifty percent, after widespread use of the antibiotic to control an outbreak of methicillin-resistant Staphylococcus aureus have been described, for example by M. A. Miller et al. MA, Dascal A, Portnoy J, Mendelson J. “Development of mupirocin resistance among methicillin-resistant Staphylococcus aureus after widespread use of nasal mupirocin ointment.”

Infect Control Hosp Epidemiol (1996 December), 17(12):811-3.

M. L. Embleton et al. in “Selective lethal photosensitization of methicillin-resistant Staphylococcus aureus using an IgG-tin (IV) chlorin e6 conjugate.” J Antimicrob Chemother 2002 December; 50(6):857-64 and “Development of a novel targeting system for lethal photosensitization of antibiotic-resistant strains of Staphylococcus aureus.” Antimicrob Agents Chemother 2005 September; 49(9):3690-6 describe phototherapeutic treatments employed to kill antibiotic-resistant S. aureus. According to Embleton et al. 2002, a conjugate comprising an antibody linked with tin(IV) chlorine e6 is employed to bind a photosensitizer to the outer surface of the bacterial cell wall. Apparently, the method of Embleton et al. utilizes the ability of some strains of MRSA to express the IgG binding protein, protein A. Embleton et al. state that this method can be used to prevent the spread of the tested strain of antibiotic-resistant S. aureus., known as EMRSA-16. However, no prevention method is described. Furthermore, Embleton et al.'s method requires a complex and presumably expensive conjugate.

The above-described problems of controlling the spread of methicillin-resistant Staphylococcus aureus are illustrative of the general problem of controlling the spread of infectious microorganisms that are capable of developing resistance to antibiotics and which can reside in carriers who are asymptomatic for infection with the microorganism.

There is accordingly a need for a new method of controlling spread of such potentially resistant, carrier-borne infectious microorganisms. The foregoing description of background art may include insights, discoveries, understandings or disclosures, or associations together of disclosures, that were not known to the relevant art prior to the present invention but which were provided by the invention. Some such contributions of the invention may have been specifically pointed out herein, whereas other such contributions of the invention will be apparent from their context. Merely because a document may have been cited here, no admission is made that the field of the document, which may be quite different from that of the invention, is analogous to the field or fields of the present invention.

### BRIEF SUMMARY OF THE INVENTION

The present invention provides, in one aspect, a method of controlling the spread of an infectious microorganism in a group of subjects, the subjects being asymptomatic for infection with the microorganism, the method comprising applying a dose of microorganism-reducing light to each anterior nasal cavity of each subject in the group to reduce any population of the infectious microorganism present in the respective anterior nasal cavity of the subject and, optionally, applying a colorant to the anterior nasal cavity to sensitize any microorganism present in the anterior nasal cavity to the microorganism-reducing light.

The subjects in the group treated can comprise both asymptomatic carriers of the microorganism infection and asymptomatic subjects, the latter being individuals who are uninfected with the microorganism. The subjects can be colonized with the microorganism and asymptomatic, not colonized with the microorganism and asymptomatic or colonized with the microorganism and symptomatic. For symptomatic subjects, relatively radical therapies may be indicated for use in conjunction with, or in place of, the method of the invention.

In another aspect, the invention provides a light applicator system for applying light to the interior nasal anatomy of a treatment subject. The light applicator system can comprise a fiber optic tip, a support-mounted light-emitting diode, or other suitable light output member, to deliver light within the nose and a hollow light-transmissive nasal dilator insertable through a naris of the treatment subject. The fiber optic tip, if employed, can have any one of a variety of suitable shapes, for example cylindrical, spherical, conical or tapered.

The nasal dilator can dilate the nose and the local light source can be received into the hollow interior of the nasal dilator to deliver light to the interior nasal anatomy through the nasal dilator. In one embodiment of the invention, the light output member can be accommodated within the nasal dilator.

Usefully, the nasal dilator can facilitate uniform distribution of light over internal nasal tissue surfaces that may harbor target microorganisms, for example the mucous membranes with the nasal vestibule at the entrance to each nostril. To this end, the nasal dilator can dilate and possibly distend the more flexible nasal tissue, in some cases unfolding folds to provide light access to surfaces that would otherwise lie in the shadows or be hidden from illumination.

The light applicator system can comprise a hand piece supporting the light output member within the nasal dilator. Optionally, the hand piece can include a light diffuser extending around the light output member within the nasal dilator.

In one embodiment of the invention, the light output member comprises an optical fiber and the hand piece is removably attachable to the optical fiber.

### BRIEF DESCRIPTION OF THE SEVERAL VIEWS OF THE DRAWING

Some embodiments of the invention, and of making and using the invention, as well as the best mode contemplated of carrying out the invention, are described in detail herein, by way of example, with reference to the accompanying drawings, in which like reference characters designate like elements throughout the several views, and in which:

FIG. 1 is a schematic view of one embodiment of a nasal light applicator system useful in the practice of the invention;

FIG. 2 is a schematic view of the nasal light applicator system shown in FIG. 1 being used to apply light within the nostril of a subject according to an embodiment of the invention;

FIG. 3 is a schematic view of an embodiment of a unit dosage liquid colorant applicator useful in the practice of the invention;

FIG. 4 is a schematic view of an embodiment of selective dosage liquid colorant applicator useful in the practice of the invention;

FIG. 5 is a schematic perspective view of an embodiment of a nasal light applicator according to the invention, showing the exterior of a hand piece component of the applicator;

FIG. 6 is an enlarged view of the nasal light applicator shown in FIG. 5, showing some internal structure thereof; and

FIG. 7 is an enlarged view of a layer of internal nasal skin infected with the methicillin-resistant Staphylococcus aureus bacterium.

### DETAILED DESCRIPTION OF THE INVENTION

The invention provides methods of controlling the spread of a microorganism infection among the subjects in a group. The method comprises applying light to decolonize, or attenuate, populations of S. aureus and other microorganisms resident in the nasal cavities of asymptomatic carriers.

In one embodiment the inventive method comprises application of light energy to each nasal vestibule of each subject in a group of subjects to decolonize or reduce any populations of microorganism resident in the nasal vestibule.

In some cases, mildly or moderately symptomed carriers can be treated by a method according to the invention, if desired, and if medically appropriate. In such cases, if the subject is non-critical, the method of the invention can be used alone, as the sole means of treatment of the invention, or can be used in conjunction with other treatments, for example antibiotic therapy. A colorant can also be employed to facilitate the absorption of light radiation by the target microorganisms, if desired.

The inventive control method can comprise applying a liquid colorant to the anterior nasal cavity of the subject, inserting a light transmissive nasal dilator through a naris of the subject to dilate the nostril of the subject, inserting a light output member into the nasal dilator and activating a light source to deliver light through the nasal dilator to the anterior nasal cavity of the subject. The light output member can comprise a fiber optic tip communicating with a remote light source, a support-mounted light-emitting diode, or other suitable light source.

The inventive control method can include the applying of a colorant to the anterior nasal cavity to photosensitive the target microorganism. Colorant application can comprise crushing a colorant applicator containing a frangible capsule of colorant fluid and applying colorant to the nasal cavity with the colorant applicator.

The term “light” is used herein to include visible wavelength radiation, as well as near-visible infrared radiation or ultraviolet radiation, useful for the purposes of the invention, in the range of from about 200 nm up to about 1200 nm in wavelength. Monochromatic or polychromatic light sources can be employed. Other radiant energies, such as heat or RF energy, can be included with the applied light energy radiation, provided they do not cause adverse side effects, for example undue heating of tissue. In one embodiment of the invention, at least 50 percent of the applied radiant energy is visible or near infrared light.

Use of light energy is known for example in photodynamic therapy wherein light energy converts a photosensitive drug precursor to a therapeutic agent for treatment of malignant and other diseases, including diseases of the upper gastrointestinal tract for example as disclosed by M. H. Johnston in “Technology insight: ablative techniques for Barrett's esophagus—current and emerging trends.” Nat Clin Pract Gastroenterol Hepatol 2005 July; 2(7):323-30. Photoactive therapies have also been described for combating bacterial infectious disease. Also, Krespi et al in U.S. Patent Application Publication No. 2005/0107853 disclose the use of light in broad-spectrum treatments of infections of the sin θ-nasal tract for example chronic rhinosinusitis. Target sin θ-nasal surfaces can be treated with a photosensitizing agent such as an oxidizing agent or a stain, for example, methylene blue or toluidine blue. While believed useful for its intended purpose, the Krespi publication does not disclose a method for controlling the spread of an infectious organism which comprises treating a group of subjects some or many of whom may be asymptomatic nor is there any disclosure of the suitability or otherwise of the methods described in the Krespi publication for treating asymptomatic carriers or noncarriers of a specific antibiotic resistant infection.

In this model there is no trauma to the host tissue because the photoactive agent binds selectively to the bacteria. Low-energy light is used that is ordinarily harmless and can only cause damage to the photosensitized bacterial cells. Heretofore, photoactive and light-radiation-based methods have been employed therapeutically to treat patients presenting disease symptoms. In contrast, the methods of the present invention can be employed prophylactically to control the spread of an infection to other people, for example the subjects in a target group of people having a common characteristic that could be associated with the spread of infection, such as proximity, common use of a facility or common use of a vehicle including a bus, train or subway car.

The methods can be employed to control the presence, colonization, early infection or spread of the Unlike known photoactive and radiation-based methods intended for the treatment of symptom-presenting individuals, the methods of the present invention are applied to a group of subjects including, or consisting of individuals who are asymptomatic for the particular microorganism infection, the spread of which is being controlled.

Some subjects in the target group may be not only asymptomatic but also free of infection with the target microorganism while others are asymptomatic carriers. Thus, the treatment group can include non-carriers who are free of the infection but who nevertheless receive a treatment pursuant to the invention. Some such individuals may be completely healthy or may have symptoms or conditions unrelated to infection with the target microorganism. In this way, possible infection in incipient carriers can be mitigated and effective control of the spread of the infection can be maintained.

The microorganism infection can spread, or be transmitted by a variety of different routes including, but not limited to, person-to-person infection, airborne infection, self-infection, for example via surface contamination from the hand to nose, surface contamination through cracked or broken skin.

Unlike use of antibiotics, such treatment of asymptomatic uninfected subjects can be acceptable employing the methods of the invention because these methods are generally relatively innocuous and can be carried out with few, if any harmful side effects. Thus the invention includes a simple, treatment of healthy subjects. The light dosages and frequencies employed in the method of the invention can be selected to avoid tissue damage, irritation or pain and are not considered likely to cause long term effects. Examples of such dosages and frequencies include the particular regimens described herein.

If desired, the light energy can be controlled to be sufficient to effect a microorganism count reduction, or decolonization, of at least about 50 percent, optionally about 80 percent, 90 percent or higher, per treatment, desirably without causing physiological damage, pain or discomfort to the patient or subject.

If desired, screening can be employed to determine the infection status with regard to the target microorganism of one or more individuals in the group. However, screening is not essential to the methods of the invention. Furthermore, the invention includes embodiments where a light treatment is applied to one or more of the subjects in a group, or even to all the subjects in a group. Carrying out the treatment method of the invention as a method, without screening, can be economical and efficient and can avoid delays. Time efficiency may be of particular importance in some circumstances, for example in treating hospital visitors at popular visiting times.

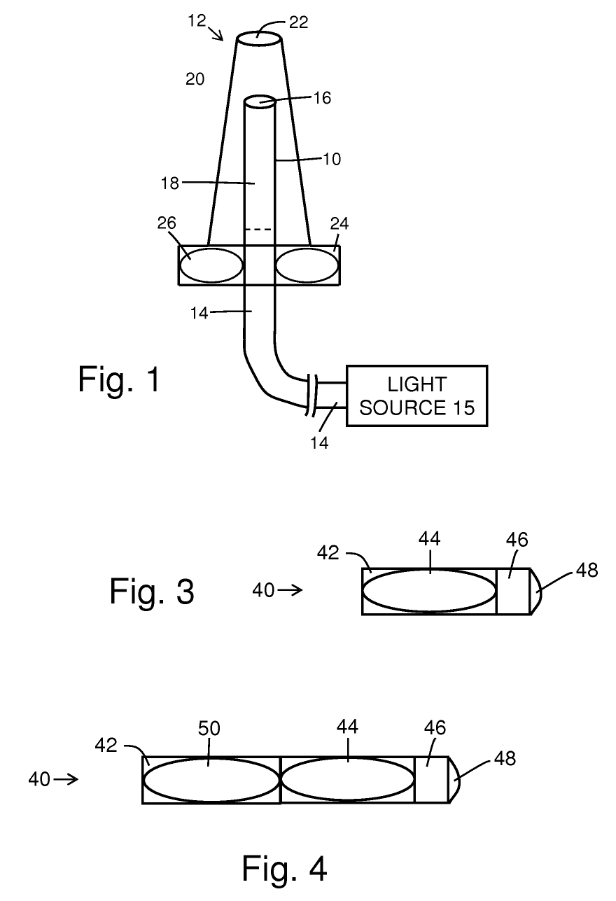

Referring now to FIGS. 1-2, the nasal light applicator system shown comprises a light output member 10 to deliver light at the point of treatment and a novel hollow light transmissive nasal dilator 12. Nasal dilator 12 can be employed to sheath fiber optic 10 and enhance the application of light from light output member 10 to the target internal nasal anatomy. Nasal dilator 12 and light output member 10 are cooperative to provide an efficient system for delivery of treatment light to the nostrils of a subject, for the purposes of the invention, and can be used for other purposes, if desired, as will be apparent to those skilled in the art in light of this disclosure.

Light output member 10 can comprise a fiber optic tip connected by optical fiber 14 to a suitable light source 15, such as one of the low-powered visible light or near-infrared lasers (not shown) that are described herein. The fiber optic tip can have opaque side walls and deliver light only from its end face 16. Alternatively, the fiber optic tip may have an end portion 18 with a light-transmitting side wall to distribute light laterally from the fiber optic tip as well as distally from end face 16. End portion 18 can have any suitable length, for example from about 2 mm to about 50 mm, or from about 15 to about 25 mm. End portion 18 can be a light diffuser, if desired. Insertion of end portion 18 through one of a subject's nares and into a nostril can help distribute light to the interior surfaces of the nose where target microorganisms may reside.

Alternatively (not shown) light output member 10 can comprise one or more light-emitting diodes (“LEDs”) having suitable output characteristics mounted on a rod-like or other suitable support member. The LEDs can be powered and switched by a remote unit, similarly to light source 15 or a small battery-powered handheld switched power supply can be provided.

The fiber optic tip can have any one of a variety of shapes including cylindrical, spherical, flared, tapered or frusto-conical and may terminate in a cup-like or ball-like diffuser or reflector, if desired. Other suitable shapes and terminations for the fiber optic tip will be known or become known to those skilled in the art, or will be apparent in light of this disclosure.

Nasal dilator 12 can serve to facilitate even distribution of the applied treatment energy to the tissues within a nasal cavity. Dilation can expose otherwise masked, obscured or hidden tissue surfaces and can also help define or provide an enlarged volume to accommodate light output member 10 in an enhanced orientation relative to the tissue to be treated.

Nasal dilator 12, in the embodiment shown, comprises a light-transmissive, frustoconical or tapered portion 20 which has a closed distal end 22, the smaller end of nasal dilator 12. Distal end 22 desirably is sized and shaped for easy and comfortable insertion into a subject's nose. For this purpose, distal end 22 can be approximately flat, but free of sharp angles or edges, or can be outwardly convex, having a configuration like the nose of a bullet. If desired, distal end 22 can comprise an internal convex reflector to redistribute light traveling distally of the fiber optic tip to radiate in lateral directions.

Proximally, nasal dilator 12 comprises a ring-like base portion 24 integral with light transmissive portion 20. Base portion 24 can also be light-transmissive, but need not be: it can be opaque, if desired.

Light output member 10 can be received into the interior of tapered portion 20, as shown in FIG. 1. Optionally, a donut-ring resilient retainer 26 can be located in base portion 24 of nasal dilator 12 to hold light output member 10 in place. Retainer 26 can frictionally engage light output member 10 to permit light output member 10 to be longitudinally positioned relative to nasal dilator 12. Nasal dilator 12 can readily be manually gripped and positioned by base portion 24 enabling tapered portion 20 to be inserted into a subject's nostril for the application of light energy thereto.

In another embodiment of the invention the light applicator system comprises multiple nasal dilators 12 having different sizes to fit treatment subjects with nostrils of different sizes. Up to five different sizes can be adequate for a wide range of subjects, including children.

Conveniently, nasal dilator 12 is of one-piece construction and is formed of a suitable transparent or translucent light-transmitting material, for example glass or a high-impact plastic. Desirably, tapered portion 20 of nasal dilator 12 is transmissive for the peak energy wavelength or wavelengths of light output by laser light source 15. Tapered portion 20 of nasal dilator 12 can be clear and transparent with high light transmissivity at relevant wavelengths, or alternatively, may be a diffuser, if desired, for which purpose it can optionally be frosted. Tapered portion 20 of nasal dilator 12 can alternatively be a filter, filtering out one or more undesired wavelengths and transmitting desired wavelengths. One embodiment of the invention employs a glass nasal dilator 12 of sufficiently durable construction to be sterilized and reused for treating more than one subject. Other sterilizable materials can be employed, if desired. In another embodiment of the invention, nasal dilator 12 is disposable.

The invention includes embodiments (not shown) of the nasal light applicator system suitable for treating both a subject's nostrils simultaneously. Such embodiments include a pair of nasal applicators and a pair of light output members, one member of each pair being intended for each nostril. Either or both pairs of nasal applicators and light output members can be formed as an integral dual unit, if desired. In one embodiment of the invention, the pair of nasal applicators and pair of light output members are constructed as an integral unit. Also, if desired, a nasal clip attaching to the septum, or other suitable means such as ties, straps or the like can be provided to support the light applicator system in place on a subject for self-administration or other purposes.

In a further embodiment of the invention, the light applicator system and light source are embodied in a single

Referring now to FIG. 2 in use, the light applicator system constituted by light output member 10 and nasal dilator 12 can be inserted into a subject's nostril 30 with end face 16 of nasal dilator 12 passing through naris 32 into the interior of nostril 30. Desirably, tapered portion 20 is dimensioned to dilate the nostril as it is inserted to promote distribution of light to the interior nasal surfaces. A nasal dilator 12 of suitable size can be selected or provided for effective nostril dilation without undue discomfort to the subject being treated.

Nasal dilator 12 can be seated in the nostril and then light output member 10 can be inserted within nasal dilator 12, with the assistance of retainer 26, if desired. Alternatively, nasal dilator 12, with light output member 10 appropriately positioned inside, can be inserted into the subject's nostril 30 as an assembly.

Light source 15 is then activated to apply a predetermined light energy dosage for a predetermined period of time through optical fiber 14, light output member 10 and nasal dilator 12 to the interior tissues and mucous membranes of the subject's nostril 30. The configuration and arrangement of the light applicator system promotes distribution of the light energy to the various internal nostril anatomies and facilitates the reaching of cavities and folds that may harbor target microorganisms.

One such cavity which can usually be reached and treated with the illustrated light applicator system of the invention is the nasal vestibule 34, also known as the anterior nares, or anterior nasal cavity. There is one nasal vestibule 34 on each side of the nose. As illustrated, the nasal vestibule 34 is typically an anterior cavity in the tip of the nose just inside each naris. Nasal vestibule 34 is often dark, moist, hairy and readily accessed by airborne bacteria, providing favorable conditions for the bacterial colonization. It is a location where target microorganisms may often be resident and can usually be effectively treated by the methods of the invention.

Nasal vestibule 34 can be a harboring structure for carrying MRSA or other target microorganisms. Microorganisms located further back in, or up, the nose, may be subject to ciliary motion which clears the bacteria or other microorganism to the back of the nose and thence into the gastro-intestinal tract where they are neutralized. In the nasal vestibule 34 there is no ciliary motion and the typically thick, glue-like mucous can provide a favorable environment to house and possibly propagate the microorganism.

Different subjects have different nasal vestibule anatomies. These may be as diversified as the appearance of the human nose is varied. Flattened, bulbous, pointed, long, short, small, large, fleshy, bony and other noses can all have different nasal vestibules 34. The internal structure of a given nasal vestibule can have more or fewer open surfaces, folds, grooves, cavities, bumps, crevices and the like that may protect target microorganisms from applied radiant light.

As described, nasal dilator 12 can facilitate uniform distribution of light over internal nasal tissue surfaces that may harbor target microorganisms, for example the mucous membranes within the nasal vestibule at the entrance to each nostril and also the nasal septum surfaces. In general, it is desirable that all or most microorganisms in the colony are directly reached by and receive the applied light dosage. To this end, nasal dilator 12 can dilate and possibly distend the more flexible nasal tissue, in some cases smoothing or straightening folds to expose the anatomy to the light. In some cases, tissue surfaces that might otherwise lie in the shadows or be hidden from illumination can be effectively illuminated by employing nasal dilator 12. Cylindrical, conical, frustoconical or other shapes of nasal dilator 12 can be useful for this purpose.

Referring now to FIG. 3, a liquid colorant applicator 40 comprises a resilient open-ended cylindrical vial 42 containing a frangible capsule 44 filled with colorant liquid, for example methylene blue, rose Bengal, or another suitable dye or stain. Frangible capsule 44 can have a defined volume providing a unit dosage. The dosage can be in any desired concentration, for example an aqueous or other solution of from about 0.01 to about 0.1 percent by weight of the colorant. A selection of colorant applicators 40 in different dilutions can be provided to a medical practitioner or treatment technician, if desired.

The open end of vial 42 is closed by a porous plug 46 of gauze material fabric or other suitable porous material. Colorant applicator 40 can provide an easy-to-use unit dose of liquid colorant in a known and controlled manner. Colorant applicator 40 can have any suitable liquid capacity, for example from about 0.1 ml to about 10 ml.

Alternatively to gauze, pursuant to the invention, porous plug 46 can comprise a porous resilient material to better seal vial 42 and prevent leakage, for example a reticulated polyurethane, porous natural or synthetic rubber, or other porous synthetic polymeric material. Porous plug 46 has a projecting tip 48 which can serve as a dauber or swab. Tip 48 can have any suitable shape, for example, cylindrical, cylindrical with a rounded end, mushroom-shaped and so on. The user squeezes vial 42 to break frangible capsule and release the liquid colorant. The liquid colorant can then be dispensed through porous plug 46 and applied to the surfaces to be treated using tip 48 as a dauber. Vial 42 may be sized to permit tip 48 access to interior nasal surfaces.

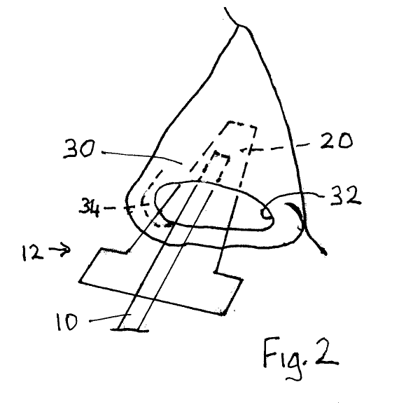

As shown in FIG. 4, colorant applicator 40 can comprise a second frangible capsule 50. Desirably, vial 42 provides a fluid pathway past intact capsule 44 for fluid from capsule 50 to reach porous plug 46 and be dispensed. Capsules 44 and 50 can be selectively crushed by the medical practitioner or other user to dispense either of the contained liquids individually or both liquids together, providing a range of convenient colorant dispensing options to the practitioner. For example, capsule 44 can contain one useful concentration or methylene blue, toluidine blue or other suitable colorant, and capsule 50 can contain deionized or distilled water, providing a choice of dilutions of the colorant. Another embodiment of liquid colorant dispenser 40 comprises a third capsule in line with capsules 44 and 50 increasing the range of concentration choices to three, or providing other options. One or more further frangible capsules, in any of various configurations can be added if desired, to further increase the options available.

If desired, the method of the invention can be performed on at least one subject in the treatment group without screening for the presence of the microorganism infection in the anterior nasal cavities of the subject.

Any suitable group of subjects can be treated. Generally, the members of the group will have something in common that brings them into contact with one another, or with a contaminated surface that may be present in a facility such as a hospital or office or in a vehicle. The common characteristic could, for example be use of the same facility or vehicle or the like. Communal contact raises the risk of contagion with the microorganism, should any member of the group be a carrier of the microorganism.

Some examples of groups of subjects that can be treated comprise all users of a facility, daily users of a facility, overnight users of a facility, residential users of a facility, casual users of a facility, visitors to a facility, users of a facility having close contact with other users of a facility, patients of a medical facility, in-patients of a medical facility, out-patients of a medical facility, overnight patients at a medical facility, high risk patients of a medical facility, staff of a medical facility, patient-contact staff of a medical facility, visitors to the medical facility or animals, for example pets, that are capable of harboring the microorganism infection with which animals users of a facility can have close contact, or any two or more of the foregoing groups.

Infection transmission control methods according to the invention can be employed in universal surveillance methods wherein all of a high risk population using a facility or all users of the facility are included in a treatment group.

The facility used can comprise a medical facility, for example, a hospital, a hospital ward, an emergency room, a specialist hospital unit, an intensive care unit, a dialysis unit, an in-patient clinic, an out-patient clinic, a nursing home, a long-term care facility, a mobile medical unit or a field medical unit. Other possible facilities used can include a school, pre-school or kindergarten, a gymnasium, a sports team's facilities, an office, a prison or any other community group facility subject to constant use by individuals who may contaminate or be contaminated by one another or a common surface, object or material with which they come into contact.

In one embodiment of the invention, the microorganism is an antibiotic-resistant microorganism and the microorganism-reducing radiation is selected to reduce the population of antibiotic-resistant microorganisms. For example, the target microorganism can be or comprise methicillin-resistant Staphylococcus aureus.

Some examples of microorganisms that can be targeted are antibiotic-resistant microorganisms selected from the group consisting of Staphylococcus aureus, alphahemolytic streptococci, Streptococcus pneumoniae, Haemophilus influenzae and coagulase-negative Staphylococci.

Some further examples of possible target microorganisms are certain fungi, including fungi selected from the group consisting of non-resistant or antifungal-resistant strains of aspergillus, candida and penicillium families, mycoplasma, alternaria and Chlamydia. Other suitable target microorganisms will be known or apparent to those skilled in the art.

The energy and duration of the microorganism-reducing light dose desirably is sufficient to reduce the microorganism population and insufficient to cause tissue damage or pain to the treated subject.

Suitable light energy can be provided in the inventive treatments by employing a laser, laser diode, light-emitting diode or other light source capable of outputting light at a wavelength or wavelengths in a range of from about 200 nm to about 1500 nm. If desired the light energy may be output in a range of from about 400 nm to about 1200 nm. In one embodiment of the invention, at least 80 percent of the energy is output in a visible wavelength range of from about 400 nm to about 700 nm. In some embodiments of the invention, the light energy does not include, or substantially excludes ultraviolet wavelengths. For example in these embodiments, no more than about 10 percent, desirably no more than about 5 percent, or 1 percent, of the light energy is at wavelengths below about 380 nm.

In embodiments where a colorant is employed, the method can use a laser source to generate the attenuating radiation which supports output of light radiation at a wavelength in a range of from about 400 nm to about 700 nm, the colorant and radiation wavelength being selected for absorption of the radiation by the colorant. For example the colorant can be methylene blue or toluidine blue and relatively red or orange, complementary light of wavelength of about 630-660 nm may be employed. Using rose Bengal, complementary wavelengths of about 400-500 nm can be employed.

In some embodiments of the invention, no colorant is employed. Useful treatments for these embodiments can employ a laser source capable of generating infrared light at a wavelength in a range of from about 850 nm to about 950 nm.

The microorganism-reducing light can be applied at any suitable energy level and duration, which parameters can be determined by routine experimentation. One example is at an energy level of from about 1 mW to about 200 mW for a duration sufficient to deliver from about 0.2 to about 20 Joules.

In another example, the microorganism-reducing radiation is applied at from about 10 mW to about 100 mW for a duration sufficient to deliver from about 2 to about 10 Joules.

Treatment of subjects can be effected by a medical professional or other suitable individual, employing any suitable light energy-generating system. For example, a laser having a maximum power input of about 3 watts and supporting light output of from about 900 to about 940 nm can be employed. The laser can be used with a SMA connector and an optical fiber having a diameter of about 400 to about 800 micron, for example about 600 micron. The optical fiber can have any suitable tip for example a tip having an external diameter of about 1.5 mm, and a diffuser of length of about 10-20 mm. A fiber length of 0.5 to 1.5 meters can be convenient. Some other specifications can comprise an optical fiber of from about 100 to about 1200 micron diameter, a tip having an external diameter of from about 0.5 to about 2 mm, and a diffuser of length of from about 3 to about 30 mm.

A useful treatment duration can be in the range of from about 30 seconds to about 15 minutes. The invention includes embodiments wherein the treatment duration is from about 1 minute to about 10 minutes, for example from about 2 to about 5 minutes.

The method of the invention can comprise providing a subject with a treatment device to generate the treatment radiation to enable the subject to self-administer the treatment in place of or as an adjunct to professionally applied treatment. An example of one useful device for this purpose is a BioNase (trademark) phototherapy system supplied by Syro Technologies R & D Ltd., of Jaffa, Israel. This product is a pocket-sized unit with dual output outputting red light at 6 mW per nostril timed for a treatment duration of about 4.5 minutes. Such a device can include a nasal clip, clampable to the septum, to support the device for self-administration.

In one embodiment of the invention, the treatment is carried out to effect a microorganism count reduction of at least about 50 percent. In another embodiment, the treatment is carried out to effect a microorganism count reduction of at least about 80 percent. The treatment can be carried out to effect a microorganism count reduction of at least about 90 percent or of at least about 95 percent. The invention includes embodiments wherein the microorganism count reduction effected is higher than 95 percent, for example in the range of from 98 to 100 percent. A desired reduction can be effected in a single treatment, or as a result of multiple treatments having cumulative effect.

The treatment can be repeated with any desired frequency, for example, within about 1 to 7 days, or upon every usage of a facility frequented by a particular subject.

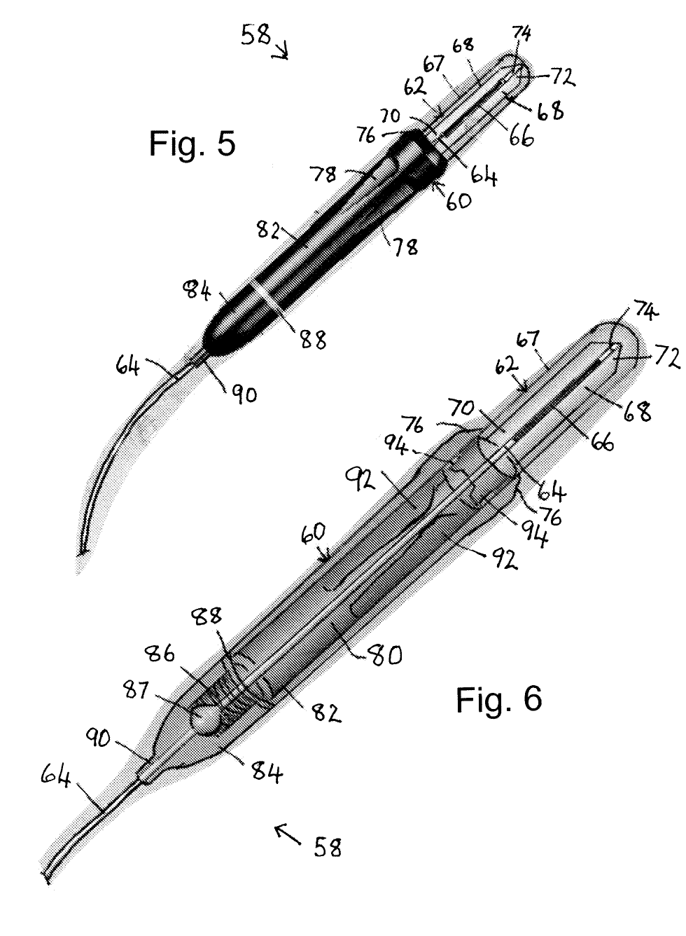

Referring to FIGS. 5 and 6, the nasal light applicator here shown, reference 58, comprises a generally cylindrical hand piece 60 on which a light tube 62 is supported as a distal extension thereof. Nasal light applicator 58 further comprises a sheathed optical fiber 64 which extends longitudinally through hand piece 60 and into light tube 62 and a light source (not shown), for example a laser, a laser diode, a light-emitting diode, a gas discharge lamp, a flash lamp, an intense pulsed light or another suitable light source, to supply light to optical fiber 64 in a controllable manner.

Hand piece 60 can be embodied as a disposable unit, if desired which can be dedicated to a single patient or other treatment subject. Some embodiments of hand piece 60 are releasably attachable to optical fiber 64, so that, if desired, each patient can be treated with a new or dedicated unit embodying essentially all the surfaces likely to be contacted by the patient or the physician, technician or other user. If desired, hand piece 60 can be provided in a variety of sizes for patients with different anatomies. The dimensions and other structural characteristics of nasal light applicator 58 can also be varied to provide units of different functionality, for example a tip-of-the-nose probe, a deep nasal probe or other desired nasal light applicator. Individual hand pieces 60 can be sterilized and sealed in their own wrapper, if desired.

Optical fiber 64 has a portion within light tube 62 from which the sheathing has been removed to provide a length of exposed fiber 66 from which light transmitted along optical fiber 64 can radiate laterally, and in other directions, to be applied to the treatment site.

Light tube 62 comprises a transparent or translucent outer cover 67 and a cylindrical diffuser 68 disposed within outer cover 67 and around exposed fiber 66. Cylindrical diffuser 68 can have a transparent or translucent appearance and can be formed of frosted or whitened or other suitable diffusing material. In one embodiment of the invention, cylindrical diffuser 68 completely surrounds exposed fiber 66 to diffuse light emitted from exposed fiber 66 and scatter it laterally of light tube 62. If desired, outer cover 67 can be disposable. One or more outer covers 67 can be packaged with hand piece 60, if desired, or they can be packaged separately.

Both outer cover 67 and cylindrical diffuser 68 desirably have good light transmissivity for the treatment light and can be formed of any suitably transmissive material, such as acrylic or polycarbonate plastic, or glass. Optionally, cylindrical diffuser 68 can have a light transmissivity which is limited to a selected waveband and can, if desired, be a light filter, for example an orange or red filter.

Cylindrical diffuser 68 can be supported in any suitable manner. For example, cylindrical diffuser 68 can have a base portion 70 supported by hand piece 60. Optionally, cylindrical diffuser 68 can have a tip portion 72 which tapers distally and supports the distal tip 74 of optical fiber 64 and can, if desired have a small aperture or recess to receive and locate distal tip 74 of optical fiber 64. If desired, for example for structural stability, tip portion 72 of optical fiber 64 can be sheathed. Also, base portion 70, or other relevant hand piece structure, can be reflective to block light traveling proximally and redirect it distally.

In one embodiment of the invention, a shield or mask (not shown) is provided around, or partially replacing, cylindrical diffuser 68 to limit the output light pattern to a desired window, for example, a selected radial angle.

In some embodiments of the invention, light tube 62 is dimensioned to be receivable into a patient's nostril and can be provided in different sizes according to an intended patient's anatomy. To this end, hand piece 60 can include a number of outer covers 67 for light tube 62 which have different diameters and lengths. Some exemplary lengths of outer cover 67, measured to shoulder 76 of hand piece 60, can be in a range of from about 5 mm to about 30 mm, desirably from about 10 mm to about 20 mm. Some exemplary diameters of outer cover 67, can be in a range of from about 3 mm to about 20 mm, desirably from about 5 mm to about 10 mm.

Usefully, outer cover 67 can be configured and dimensioned to function as a nasal dilator, for example by selecting its dimensions, in relation to a particular patient so that the patient's nostril will be appropriately dilated, as described herein, when light tube 62 is inserted into the patient's nostril. In one embodiment of the invention, the patient's nostril is significantly distended when light tube 62 is sufficiently inserted to illuminate the interior of the nostril, including the nasal vestibule.

Externally, the embodiment of hand piece 60 shown has an ergonomic structure enabling it to be easily and conveniently manipulated, for example in the manner of a pen or pencil, by gripping it between the thumb and forefingers. Other suitable shapes or configurations of hand piece 60 will be apparent to a person of ordinary skill in the art. Some optional external structural features of hand piece 60 include a smooth beveled shoulder 76, recesses 78 and elongated, slender overall proportions.

Shoulder 76 of hand piece 60 can abut the patient's nostril and prevent over-insertion of light tube 62 into the nostril, in some cases. Optionally, and depending upon the particular patient, shoulder 76 and light tube 62 may adequately dilate the patient's nostril without use of a separate instrument such as nasal dilator 12. Recesses 78 can comprise small depressions suited to be engaged by the tips of a user's thumb and/or fingers, to facilitate control and manipulation of hand piece 60.

Internally, hand piece 60 has a hollow longitudinal cavity 80 to accommodate optical fiber 64 to which hand piece 60 can be secured by suitable clamping or other means. In some useful embodiments of the invention, hand piece 60 is releasably attachable to optical fiber 64. This capability can permit the tension in the fiber to be adjusted and allow for replacement of worn or damaged fiber either by moving hand piece 60 along optical fiber 64 to a new length of fiber, or by complete replacement of the fiber. In another embodiment of the invention, hand piece 60 is permanently attached to optical fiber 64.

Various mechanical arrangements for releasably attaching hand piece 60 to optical fiber 64 will be or become apparent to a person of ordinary skill in the art. For example, handpiece 60 can be formed in two sections, namely a body section 82 and an end section 84 which are screwed together by means of threads 86, the sections meeting joining at a band 88. Screwing the sections together brings thread 86 on body section 82 into engagement with a ball clamp 87. Ball clamp 87 is resiliently compressible and has an axial opening (not shown) to receive optical fiber 64. As body section 82 and end section 84 are tightened together, the thread 86 on body section 82 bears down on ball clamp 87 compressing it around, and locking it on to, optical fiber 64 desirably without shifting optical fiber 64 axially.

Optionally, end section 84 of hand piece 60 can have a short end sleeve 90 through which optical fiber 64 can be inserted into hand piece 60. Desirably, end sleeve 90 can frictionally grip optical fiber 64 to act as a strain relief device preventing external strains being transmitted to ball clamp 82 and other downstream structures.

To further stabilize the mounting of hand piece 60 on optical fiber 64, a number, for example three or four, of circumferentially arranged and radially extending contoured guide ribs 92 can be provided in body section 82 of hand piece 60 towards its distal end. Guide ribs 92 can center optical fiber 64, and optionally can slidingly engage it with limited pressure, helping to position optical fiber 64 in light tube 62.

Outer cover 67 of light tube 62 can be attached to hand piece 60 in any suitable manner, desirably in a removable manner. For example, outer cover 67 can be a snap fit into a recess in the forward or distal end of body section 82 of hand piece 60, and can, if desired be locked in place by rotational engagement of detent such as detents 94, providing a quickly connected fitting that is easily manipulated by a busy physician or other operator.

Body section 82 and end section 84 of hand piece 60 can be manufactured in any suitable manner from appropriate materials, for example by molding from plastics materials. For example, the more complex body section 82 can be fabricated from polycarbonate or the like, and the simpler end section 84 can also be fabricated from polycarbonate or from a polyolefin, or an acrylic polymer or copolymer or the like.

In one method of use of nasal light applicator 58 a physician, an infection control nurse clinician, or other appropriate technician, or operator, unwraps a new or sterilized hand piece 60 and selects a light tube outer cover 67 of appropriate size for the patient to be treated. If necessary, an existing outer cover 67 can be removed from the hand piece 60 and one of appropriate size can be quickly snapped into place. Alternatively, the outer cover 67 of appropriate size can be fitted to hand piece 60 after the latter is assembled to optical fiber 64, which assembly is described below.

The hand piece 60 is assembled with optical fiber 64 by first unscrewing end section 82 from body section 84 of hand piece 60 to sufficiently to open ball clamp 87. Optical fiber 84, with a section of sheathing removed to provide a suitable length of exposed fiber 66 at its distal end, is then manually threaded through end sleeve 90, through ball clamp 87 and between guide ribs 92 to emerge into light tube 62 where its distal tip can be advanced to engage with tip portion 72 of cylindrical diffuser 68. End section 84 is then screwed into body section 82 to lock ball clamp 87 onto optical fiber 64 and provide a secure, integral assembly. Disassembly of hand piece 60 from optical fiber 84 can be quickly accomplished by reversing the assembly procedure. Employing some embodiments of the invention, both assembly and disassembly can readily be accomplished without the use of tools.

The proximal end of optical fiber 64 is then connected to a light source, if not already connected, and nasal light applicator 58 is ready for use.

To apply a light treatment to the patient's nostril, the physician or other user, the physician can grip hand piece 60 in one hand and gently insert light tube 62 through the naris into the patient's nostril with sufficient penetration to provide a desired field of illumination within the nostril. The depth of penetration can for example be about 8 mm to 12 mm in pediatric cases or from about 15 mm to about 20 mm in adult cases.

After insertion of light tube 62, the physician switches the light source causing light to radiate from exposed fiber 66 and diffuse through cylindrical diffuser 68 to illuminate the interior of the patient's nostril with light of the selected wavelength or waveband. For example, nostril can be illuminated coaxially and homogeneously with the illumination in the direction of the mechanical axis of the fiber being prevented. If desired, colorant can be applied inside the nostril before applying the light treatment.

In some useful embodiments of the invention the ergonomic design of hand piece 60 enables it to be conveniently gripped, permitting the physician or other holder to effect a carefully controlled insertion of light tube 62 into the patient's nostril, with fine movements to position hand piece 60, as desired for one or more light treatments, for example for treatments at different depths of penetration or different angles. Hand piece 60 is held in place in the patient's nostril for the duration of each treatment, for example for from about 1 to about 3 minutes.

When one nostril has been adequately treated hand piece 60 is moved to the other nostril and a similar treatment is performed. If desired, outer cover 67 can be removed and a new outer cover 67 can be fitted to hand piece 60 for treating the patient's other nostril to avoid cross-contaminating the nostrils. Optionally, the two outer covers 67 can be identified with respect to the nostril for which they have been used and used for the same nostril in future treatments of the particular patient. Thus, it is possible to dedicate a hand piece 60 to a particular patient and to dedicate one or more outer covers 67 to each of the patient's individual nostrils. Both hand piece 60 and the outer covers 67 can be re-used in future treatments of the patient and disposed of at the end of a course of treatment, or when no longer serviceable. A new, or newly sterilized, hand piece 60 is employed for the next patient.

By employing a slightly oversized outer cover 67, stretch the internal skin of the nostril can be stretched as light tube 62 is introduced into the nostril, exposing the hair within the nostril and baring the inner lining the skin of the nostril, to facilitate illumination of the inner lining of the nasal skin for desired period of time. A tight fit of light tube 62 within the nostril can help keep the tip of the applicator instrument in a fixed position.

In summary, nasal light applicator 58, as illustrated in FIGS. 5-6, can be embodied as a disposable unit to be used for a single patient to control contamination from patients carrying pathogenic bacteria. Since the distal end of the hand piece 60 is inserted into the patient's nostril, a disposable cover is provided which can be replaced easily between nostrils and potentially used in multiple applications in the same patient.

While nasal light applicator 58 has been described with reference to the example of the application of treatments by a medical professional, it will be understood that some embodiments of nasal light applicator 58 are suitable for home use or for self administration.

As stated, a colorant can be employed to promote absorption of light energy by the target microorganism. Usefully, the colorant can be absorbed by the target microorganism coloring it so that it absorbs light. Desirably, the colorant can lack or have only limited therapeutic or other biomedical effects other than photosensitizing the microorganism. In some cases, the colorant will be absorbed by the surrounding mucous membrane or other tissue, coloring the tissue. The invention includes embodiments wherein the effect of the colorant on the tissue in such an instance is minimal, so that biological effects of the colorant on host tissue, other than the temporary coloring, can be largely or entirely avoided.

Colorants or other photosensitizers which are absorbed through a bacterium or other microorganism cell wall and that can be photoactivated, pursuant to the invention, to kill the bacterium can be employed in the invention. Desirably, colorants that are effective at most or all stages of the cell cycle, for example methylene blue or rose Bengal, can be employed.

One or more examples of suitable colorants that can be employed can be selected from the group consisting of stains, dyes, pigments, methylene blue, rose Bengal, arianor steel blue, toluidine blue, tryptan blue, crystal violet, azure blue cert, azure B chloride, azure 2, azure A chloride, azure B tetrafluoroborate, thionin, azure A eosinate, azure B eosinate, azure mix sicc., azure II eosinate, haematoporphyrin hydrogen chloride, aluminum disulfonated phthalocyanine and chlorins.

The colorant, if employed, can be selected to cause little effect other than temporary esthetic effects, notably a coloring of the tissue to which the colorant is applied. Such coloring may be largely out of sight, within the nostril and soon dissipated or otherwise removed.

The colorant can be used in solution, for example aqueous solution, in any suitable concentration, for example a concentration of from about 0.01 percent to about 0.1 percent, by weight of the solution. For example concentrations of methylene blue in the range of from about 0.03 to about 0.06 percent are also believed to be particularly effective. It will be appreciated that other stains, dyes or colorants can be used at different concentrations according to their efficacy as known or as determined by routine experimentation.

If desired, the method can comprise screening the group of subjects to identify asymptomatic but infected subjects for treatment. Symptomatic subjects having a positive indication of infection with the microorganism can be treated with an antibiotic.

A nonlimiting example illustrating the presence of asymptomatic methicillin-resistant S. aureus(“MRSA”) carriers in the population will now be described.

### EXAMPLE 1

Nasal cultures are obtained from a total of 100 patients enrolled in a study performed under informed consent on an ambulatory patient population at a medical facility, each of whom is presenting with sin θ-nasal symptoms. In four confirmed S. aureus-colonized patients nasal skin biopsies are performed during elective nasal procedures (SMR). Related information regarding each patient's age, health status, frequency and reason for the clinic visits, and last date of hospitalization, significant medical history, antibiotic use and contacts with illness is gathered. History about any recent skin trauma, skin lesion, infection, tattooing or any drug use is also elicited. A culture method as standardized by a hospital infection control group is used on each patient. A sterile swab is inserted into the anterior part of one nostril. The swab is then rotated three times, slowly and circumferentially around the internal nostril for 4-5 seconds, applying even pressure. The swab is removed, taking care not to touch anything else and returned back into its transport sleeve. Patients with positive MRSA culture requiring hospitalization, intervention or surgical procedure are treated with mupirocin ointment and chlorhexidine washes. Mupirocin is applied twice daily for five days to anterior nares. Post treatment cultures are obtained one month later to confirm decolonization.

Results of the study show that around 40% of patients are colonized with methicillin-sensitive S. aureus(“MSSA”) and about 4% are colonized with MRSA. About half of the colonized patients had no apparent risk factors. MRSA is an isolate of the bacterium Staphylococcus aureus that has acquired genes encoding antibiotic resistance to essentially all penicillins, including methicillin and other narrow-spectrum β-lactamase-resistant penicillin antibiotics.

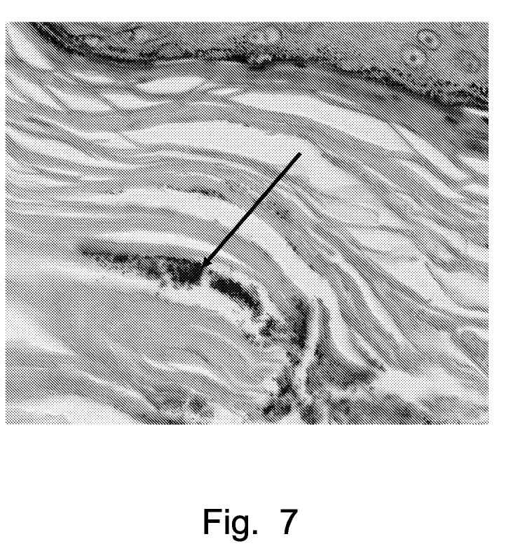

Patients carrying MRSA can be split into two groups; those who are colonized and those who are infected. Staphylococcus colonization is known to colonize skin, nares and pharynx. The anterior nares however appear to be a primary reservoir of S. aureus in humans. Intracellular bacteria located within the keratin layer of the nasal epithelium are believed to provide a sanctuary for this bacterium by protecting them from host defense mechanisms and from antibiotic treatment. This is illustrated in FIG. 7 which shows a biopsy from a patient who is an asymptomatic MRSA carrier. The protected methicillin-resistant S. aureus bacterium is indicated by an arrow. Desirably, the light energy applied in the methods of the invention has sufficient intensity to penetrate to, and destroy or attenuate, such protected microorganisms.

Nasal carriage is a risk factor for staphylococcal infections, with greater than 80% of infecting isolates believed to originate from the nose of the patient. In addition, eradication of nasal carriage can eliminate the organism from other body sites.

The foregoing detailed description is to be read in light of and in combination with the preceding background and invention summary descriptions wherein partial or complete information regarding the best mode of practicing the invention, or regarding modifications, alternatives or useful embodiments of the invention may also be set forth or suggested, as will be apparent to one skilled in the art. Should there appear to be conflict between the meaning of a term as used in the written description of the invention in this specification and the usage in material incorporated by reference from another document, the meaning as used herein is intended to prevail.

Throughout the description, where compositions are described as having, including, or comprising specific components, or where processes are described as having, including, or comprising specific process steps, it is contemplated that compositions of the present invention can also consist essentially of, or consist of, the recited components, and that the processes of the present invention can also consist essentially of, or consist of, the recited processing steps. It should be understood that the order of steps or order for performing certain actions is immaterial so long as the invention remains operable. Moreover, two or more steps or actions may be conducted simultaneously. In addition, all proportions recited herein are to be understood to be proportions by weight, based upon the weight of the relevant composition, unless the context indicates otherwise.

While illustrative embodiments of the invention have been described above, it is, of course, understood that many and various modifications will be apparent to those of ordinary skill in the relevant art, or may become apparent as the art develops, in the light of the foregoing description. Such modifications are contemplated as being within the spirit and scope of the invention or inventions disclosed in this specification.

1. A method of controlling the spread of an infectious microorganism among the subjects in a group of subjects, the group comprising subjects asymptomatic for infection with the microorganism, the method comprising applying a dose of microorganism-reducing light to each anterior nasal cavity of each subject in the group and, optionally, applying a colorant to the anterior nasal cavity to sensitize infectious microorganisms present in the anterior nasal cavity to the microorganism-reducing light.

2. A method according to claim 1 wherein the subjects in the group comprise asymptomatic carriers of the microorganism infection and asymptomatic subjects uninfected with the microorganism and optionally comprise subjects noncritically symptomatic for infection with the microorganism.

3. A method according to claim 1 wherein the dose of microorganism-reducing light is applied to each nasal vestibule of each subject.

4. A method according to claim 1 wherein the group of subjects treated comprises all users of a facility, daily users of a facility, overnight users of a facility, residential users of a facility, casual users of a facility, visitors to a facility, users of a facility having close contact with other users of a facility, patients of a medical facility, in-patients of a medical facility, out-patients of a medical facility, overnight patients at a medical facility, high risk patients of a medical facility, staff of a medical facility, patient-contact staff of a medical facility, visitors to the medical facility or animals capable of harboring the microorganism infection with which animals users of a facility have close contact or any two or more of the foregoing groups.

5. A method according to claim 4 wherein the facility is selected from the group consisting of a medical facility, a hospital, a hospital ward, an emergency room, a specialist hospital unit, an intensive care unit, a dialysis unit, an in-patient clinic, an out-patient clinic, a nursing home, a long-term care facility, a mobile medical unit a field medical unit, a school, a pre-school, a kindergarten, a gymnasium, a sports team's facilities, an office, a prison and a community group facility.

6. A method according to claim 1 comprising inserting a light-transmissive nasal dilator through a naris of the subject to dilate the nostril of the subject, inserting a fiber optic tip into the nasal dilator and activating a light source to deliver the dose of microorganism-reducing light through the fiber optic tip and through the nasal dilator to the anterior nasal cavity of the subject.

7. A method according to claim 6 comprising applying a colorant to the anterior nasal cavity, wherein the applying of a colorant to the anterior nasal cavity comprises crushing a colorant applicator containing a frangible capsule of colorant fluid and applying colorant to the nasal cavity with the colorant applicator.

8. A method according to claim 1 wherein the energy and duration of the dose of microorganism-reducing light is sufficient to reduce the microorganism population and insufficient to cause tissue damage or pain to the subject.

9. A method according to claim 1 wherein the infectious microorganism comprises one or more microorganisms selected from the group consisting of an antibiotic-resistant microorganism, methicillin-resistant Staphylococcus aureus, antibiotic-resistant Staphylococcus aureus, antibiotic-resistant alpha-hemolytic streptococci, antibiotic-resistant Streptococcus pneumoniae, antibiotic-resistant Haemophilus influenzae, antibiotic-resistant coagulase-negative Staphylococci, aspergillus, candida and penicillium families, mycoplasma, alternaria, Chlamydia, antifungal-resistant aspergills, antifungal-resistant candida and antifungal-resistant penicillium families, antifungal-resistant mycoplasma, alternaria and antifungal-resistant Chlamydia.

10. A method according to claim 9 comprising employing, to generate the microorganism-reducing light, a light source capable of outputting light at a wavelength in a range of from about 200 nm to about 1500 nm, the light source optionally being a laser, a laser diode, a light-emitting diode, a gas discharge lamp, a flash lamp or an intense pulsed light.

11. A method according to claim 1 wherein the colorant is applied and the method comprises employing a laser source to generate the attenuating light and wherein the laser source outputs light at a wavelength in a range of from about 400 nm to about 700 nm, the colorant and light wavelength being selected for absorption of the light by the colorant.

12. A method according to claim 1 wherein the colorant is not applied and the method comprises employing a laser source to generate the microorganism-reducing light and wherein the laser source outputs light at a wavelength in a range of from about 850 nm to about 950 nm.

13. A method according to claim 1 wherein the microorganism-reducing light is applied at an energy of from about 1 mW to about 200 mW for a duration sufficient to deliver from about 0.2 to about 20 Joules.

14. A method according to claim 1 wherein the microorganism-reducing light is applied at an energy intensity of from about 10 mW to about 100 mW for a duration sufficient to deliver from about 2 to about 10 Joules.

15. A method according to claim 1 wherein the method is carried out to effect a microorganism count reduction of at least about 50 percent.

16. A method according to claim 1 comprising repeating the method within about 1 to 7 days.

17. A method according to claim 1 wherein the colorant is selected from the group consisting of stains, dyes, pigments, methylene blue, rose Bengal, arianor steel blue, toluidine blue, tryptan blue, crystal violet, azure blue cert, azure B chloride, azure 2, azure A chloride, azure B tetrafluoroborate, thionin, azure A eosinate, azure B eosinate, azure mix sicc., azure II eosinate, haematoporphyrin hydrogen chloride, aluminum disulfonated phthalocyanine and chlorines and optionally is employed in aqueous solution in a concentration of from about 0.01 percent to about 0.1 percent by weight of the solution.

18. A method according to claim 1 wherein the method comprises screening the group of subjects and treating with an antibiotic subjects having a positive indication of infection with the microorganism.

19. A method according to claim 1 comprising performing the method on at least one subject in the group without screening for the presence of the microorganism infection in the anterior nasal cavities of the subject.

20. A light applicator system for applying light to the interior nasal anatomy of a treatment subject, the light applicator system comprising a light output member to deliver light within the nose and a hollow light-transmissive nasal dilator insertable through a naris of the treatment subject to dilate the nose, wherein the light output member can be accommodated in the hollow interior of the nasal dilator to deliver light to the interior nasal anatomy through the nasal dilator.

21. A light applicator system according to claim 20 wherein the nasal dilator comprises a light-transmissive tapered portion having a closed distal end and a ring-like base portion for gripping the nasal dilator.

22. A light applicator system according to claim 21 wherein the nasal dilator is of one-piece construction and is sterilizable and reusable for another subject.

23. A light applicator system according to claim 20 comprising multiple ones of the nasal dilators having different sizes to fit different subjects having nostrils of different sizes.

24. A light applicator system according to claim 20 comprising a hand piece supporting the light output member within the nasal dilator, the hand piece optionally including a light diffuser extending around the light output member within the nasal dilator.

25. A light applicator system according to claim 24 wherein the light output member comprises an optical fiber and the hand piece is removably attachable to the optical fiber.
